(19) United States
(12) Patent Application Publication
(10) Pub. No.: US 2025/0319263 A1
(43) Pub. Date: Oct. 16, 2025

(54) DEVICE FOR NASAL DELIVERY OF A FLUID PRODUCT

(71) Applicant: APTAR FRANCE SAS, LE NEUBOURG (FR)

(72) Inventor: Serge HERRY, FRANQUEVILLE SAINT PIERRE (FR)

(73) Assignee: APTAR FRANCE SAS, LE NEUBOURG (FR)

(21) Appl. No.: 18/281,185

(22) PCT Filed: Mar. 7, 2022
(86) PCT No.: PCT/FR2022/050400

§ 371 Date: Oct. 3, 2023

(30) Foreign Application Priority Data
Mar. 9, 2021 (FR) FR2102304

(51) Int. Cl. A61M 11/00

(52) U.S. Cl. CPC A61M 11/007

(57) ABSTRACT

Device for nasal delivery of fluid product having a reservoir containing a fluid dose product and a nasal delivery head with a delivery orifice having an axial cylinder with an axial height and a radial diameter. The axial height is equal to 0.6 mm, the dose volume is 50 μL and the actuation speed is 70 mm/s. For a radial diameter equal to 0.4 mm, the force of impact of the spray measured at a distance of 3 cm is less than 0.03 N and the droplet size distribution is between 50 μm and 70 μm and for a radial diameter equal to 0.5 mm, the force of the impact of the spray measured at a distance of 3 cm from said delivery orifice is less than 0.02 N and the droplet size distribution is of between 80 μm and 100 μm.

The present invention relates to a device for nasal delivery of fluid product.

A drawback of current nasal delivery devices for a fluid product relates to their compatibility with a paediatric use. Indeed, standard devices can generate sprays which have a force of impact in the nostril which is too high for newborns or young children. Typically, a standard device force generates a of impact of approximately 0.05 N to 0.1 N, which is too high for a paediatric use.

An object of the present invention is to provide a device for nasal delivery of fluid product that does not have the above-mentioned drawbacks.

More specifically, the present invention aims to provide such a nasal delivery device which avoids or limits the risks during a paediatric use.

The present invention also aims to provide such a nasal delivery device which is simple and inexpensive to manufacture and to assemble.

The present invention thus provides a device for nasal delivery of fluid product comprising a reservoir containing a dose of fluid product and a nasal delivery head provided with a delivery orifice comprising an axial cylinder having an axial height h and a radial diameter d, wherein the axial height h is equal to 0.6 mm, the dose volume is 50 μL and the actuation speed is 70 mm/s, and wherein:
for a radial diameter d equal to 0.4 mm, the force of impact of the spray measured at a distance of 3 cm from said delivery orifice is less than 0.03 N, in particular equal to 0.026 N, and the droplet size distribution is between 50 μm and 70 μm, in particular equal to 60 μm, and
for a radial diameter d equal to 0.5 mm, the force of the impact of the spray measured at a distance of 3 cm from said delivery orifice is less than 0.02 N, in particular equal to 0.019 N, and the droplet size distribution is of between 80 μm and 100 μm, in particular equal to 90 μm.

Advantageously, said reservoir comprises a stopper/piston to deliver at least some of said fluid product, said reservoir being, before actuation, sealingly blocked by said stopper/piston.

Advantageously, the device comprises a hollow insert inserted and fixed in said delivery head, said insert supporting a cannula to pierce said stopper/piston and thus connect said reservoir to said delivery orifice during actuation.

These features and advantages and others of the present invention will appear more clearly during the following detailed description, made in reference to the accompanying drawings, given as non-limiting examples, and wherein:

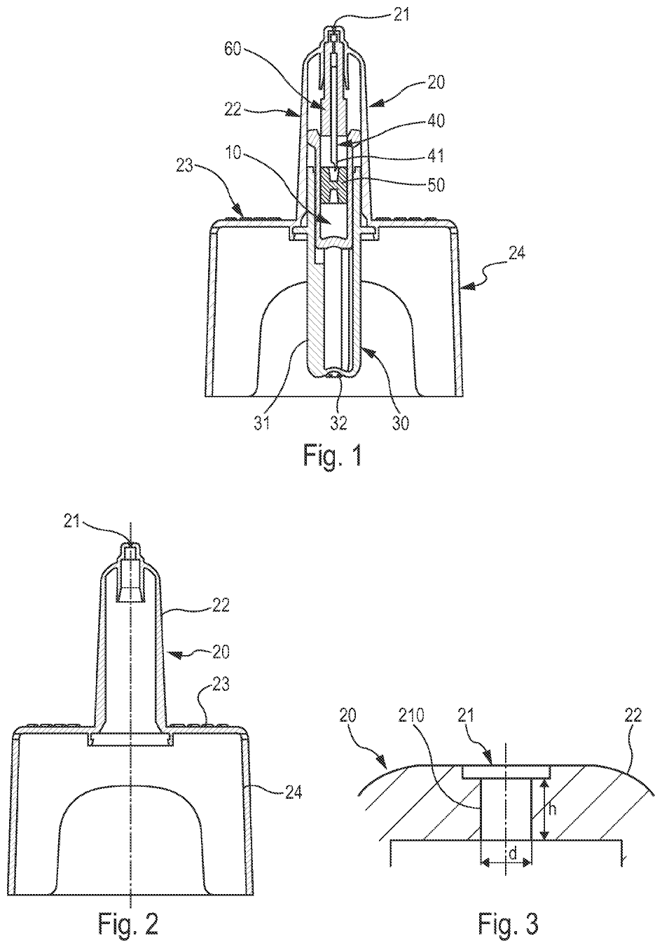

FIG. 1 is a diagrammatic cross-sectional view of a fluid product dispensing device according to one advantageous embodiment,

FIG. 2 is a detailed, cross-sectional view of the delivery head of FIG. 1, and

FIG. 3 is a schematic, enlarged view of the delivery orifice of the head of FIG. 2.

The terms “proximal” and “distal” are relative to the delivery orifice. The terms “axial” and “radial” are relative to the longitudinal central axis of the device. The terms “upstream” and “downstream” are relative to the direction of flow of the fluid during actuation. The terms “top” and “bottom” are relative to the upright position of the device shown in FIG. 1.

In the advantageous embodiment of FIG. 1, a reservoir 10 containing the fluid product to be delivered, typically a liquid, is disposed inside a body forming a delivery head 20. The delivery head 20 comprises a delivery orifice 21 oriented axially. The delivery orifice 21 serves to deliver a dose of fluid out from said delivery head 20 while the device is being actuated by a user.

The dispenser head 20 comprises an extended nasal endpiece 22 that, at its proximal axial end, includes said delivery orifice 2, and a side body 24 that is connected to said nasal endpiece 22 via a radial flange 23.

In the example of FIG. 1, a hollow insert 60 is disposed in the delivery head 20, upstream from said delivery orifice 21, said hollow insert 60 defining an expulsion channel and, in engagement with the bottom wall of the delivery head 20, a spraying profile directly upstream from said delivery orifice 21.

A hollow cannula 40 is inserted and fixed in said expulsion channel 61, said cannula 40 comprising, at its distal end, a piercing tip 41. Advantageously, the cannula 40 is forcefully sleeved in the expulsion channel.

The reservoir 10 is, in the example of FIG. 1, formed by a hollow and blind body, comprising one single opening closed by a stopper/piston 50 and containing one single dose of fluid product to be delivered in one single actuation of the device.

During actuation, the reservoir 10 is displaced axially upwards relative to the delivery head 20 and thus relative to the cannula 40. The stopper/piston 50 is thus pushed against the tip 41 of the cannula 40 in order to be pierced, which allows all or part of the fluid product contained in the reservoir 10 to be expelled.

It should be observed that the present invention may also be adapted to devices of the dual- or multidose type, wherein the reservoir contains at least one two doses of fluid to be delivered during two successive actuations of the device. Document WO 2014/147329 describes an example of a dual-dose device.

Actuation means 30 can be provided to enable the actuation of the device of FIG. 1.

In the embodiment of FIG. 1, the actuation means 30 comprise an actuation body 31 that is movable relative to the delivery head 20, said actuation body 31 engaging with said reservoir 10 so as to move it axially relative to the delivery head 20, towards the delivery orifice 21.

Operation of the device is described in detail below. In a conventional manner, the user places two fingers on the radial flange 23 that is formed on the delivery head 20, and presses with the thumb on the distal axial bottom 32 of said actuation body 31. During such actuation, the reservoir 10 is thus pushed axially towards the dispenser orifice 21, so that the cannula 40 pierces the stopper/piston 50. The contents of the reservoir 10 are thus connected to the delivery orifice 21, and the user pressing on the actuation body 31 moves the stopper 50 in the reservoir 10 so as to deliver the fluid product.

The delivery orifice 21 comprises an axial cylinder 210 having an axial height h and a radial diameter d.

According to the invention, said axial height h is approximately equal to 0.6 mm and said radial diameter d is of between 0.4 mm and 0.5 mm.

According to a first advantageous variant of the invention, the radial diameter d is equal approximately to 0.4 mm.

According to a second advantageous variant of the invention, the radial diameter d is equal approximately to 0.5 mm.

With such a geometry of the delivery orifice 21, it is possible to reduce the force of impact of the spray exiting the delivery orifice 21, typically at values less than 0.04 N, advantageously less than 0.03 N, preferably less than 0.02 N.

This reduction of the force of impact is accompanied with an increase in the droplet size distribution (DSD), which however remains within acceptable values, of between 50 μm and 100 μm, preferably of between 60 μm and 90 μm.

The table 1 below illustrates the results of comparative tests between a standard device and two devices 1 and 2 according to the present invention.

The standard device is such as shown in FIG. 1, with a delivery orifice comprising a height h of 0.72 mm, conically-shaped by narrowing towards its downstream axial end, and with a minimum diameter at the exit, at the downstream axial end, of 0.27 mm.

The devices 1 and 2 are identical to the standard device, except concerning the geometry of the delivery orifice 21.

For the device 1, the delivery orifice 21 comprises an axial cylinder having a height h of 0.6 mm and a diameter d which is constant over said height h of 0.5 mm.

For the device 2, the delivery orifice 21 comprises an axial cylinder having a height h of 0.6 mm and a diameter d which is constant over said height h of 0.4 mm.

The test method has consisted of filling the reservoir of the delivery device with water, then of actuating the device using an automatic actuation machine at an actuation speed which is realistic for the user. The water spray emitted has hit a plate located at a distance of 3 cm from the delivery orifice of the device, said plate being associated with a sensor adapted to measure the force of impact. The dose of water emitted in the form of spray was 50 μL, and the actuation speed of the machine was 70 mm/s.

This comparative test has not only measured the force of impact, but also the droplet size distribution (DSD).

TABLE 1

| | | | Actuation | Dose | | |
|---|---|---|---|---|---|---|
| | Height | Diameter | speed | volume | Force of | DSD |
| | h (mm) | d (mm) | (mm/s) | (μL) | impact (N) | (μm) |
| Standard | 0.72 | 0.27 | 70 | 50 | 0.066 | 30 |
| device | | | | | | |
| Device 1 | 0.60 | 0.50 | 70 | 50 | 0.019 | 90 |
| Device 2 | 0.60 | 0.40 | 70 | 50 | 0.026 | 60 |

The results of the comparative test make it possible to establish that at a constant dose, and at a constant actuation speed, the geometry of the delivery orifice 21 has a significant impact on the force of impact of the spray.

It is thus observed that the device 1 has the best result concerning the force of impact, namely less than 0.02 N, in particular equal to 0.019 N, with a DSD of between 80 μm and 100 μm, in particular equal to 90 μm.

The device 2 has generated a force of impact slightly greater, less than 0.03 N, in particular equal to 0.026 N, but still broadly less than that of the standard device, namely 0.066 N, and with a DSD of between 50 μm and 70 μm, in particular equal to 60 μm.

The present invention therefore provides a device making it possible to substantially reduce the force of impact of the spray in the nostril, which makes it acceptable for a paediatric use, in particular with newborns and young children.

Naturally, variants of embodiment can be considered, without deviating from the scope of the invention, such as defined by the accompanying claims.

1-3. (canceled)

4. Device for nasal delivery of fluid product, comprising:
a reservoir containing a dose of fluid product;
a nasal delivery head provided with a delivery orifice comprises an axial cylinder having an axial height and a radial diameter, and
actuator means to make it possible to actuate the device, said actuator means comprising an actuator body that is movable relative to the nasal delivery head, said actuator body engaging during actuation with said reservoir so as to move it axially relative to the nasal delivery head towards the delivery orifice,
characterised in that the axial height is equal to 0.6 mm, the dose volume is 50 μL and the radial diameter is equal to 0.4 mm, and in that, for an actuation speed of 70 mm/s, the force of the impact of the spray measured at a distance of 3 cm from said delivery orifice is less than 0.03N, in particular equal to 0.026N, and the distribution of the droplet sizes is of between 50 μm and 70 μm, in particular equal to 60 μm.

5. Device for nasal delivery of fluid product, comprising:
a reservoir containing a dose of fluid product;
a nasal delivery head provided with a delivery orifice comprises an axial cylinder having an axial height and a radial diameter, and
actuator means to make it possible to actuate the device, said actuator means comprising an actuator body that is movable relative to the nasal delivery head, said actuator body engaging during actuation with said reservoir so as to move it axially relative to the nasal delivery head towards the delivery orifice,
characterised in that the axial height is equal to 0.6 mm, the dose volume is 50 μL and the radial diameter is equal to 0.5 mm, and in that, for an actuation speed of 70 mm/s, the force of the impact of the spray measured at a distance of 3 cm from said delivery orifice is less than 0.02N, in particular equal to 0.019N, and the distribution of the droplet sizes is of between 80 μm and 100 μm, in particular equal to 90 μm.

6. Device according to claim 4, wherein said reservoir comprises a stopper/piston to deliver at least some of said fluid product, said reservoir being, before actuation, sealingly blocked by said stopper/piston.

7. Device according to claim 6, comprising a hollow insert inserted and fixed in said delivery head, said insert supporting a cannula to pierce said stopper/piston and thus connect said reservoir to said delivery orifice during actuation.
